(19) United States
(12) Patent Application Publication
(10) Pub. No.: US 2020/0107900 A1
(43) Pub. Date: Apr. 9, 2020

(54) Sterility barrier for surgical instrument table

(71) Applicant: James Cederlind, San Bernardino, CA (US)

(72) Inventor: James Cederlind, San Bernardino, CA (US)

(21) Appl. No.: 16/152,325

(22) Filed: Oct. 4, 2018

(51) Int. Cl. A61B 46/10

(52) U.S. Cl. CPC A61B 46/10

(57) ABSTRACT

The disclosure relates to a protective barrier for a surgical table containing a sterile field that may be used in a surgical facility to assist in maintaining the sterility of the sterile field. The sterile field may hold surgical instruments or equipment. The protective barrier may be efficiently stored, easily assembled by simply folding along various guides on the barrier, and enclosed one or three sides of the sterile field.

### BACKGROUND OF THE INVENTION

### 1. Field of the Invention

The invention relates to a protective barrier for a surgical instrument table containing a sterile field that may be used in a surgical facility to assist in maintaining the sterility of the sterile field. The sterile field may hold surgical instruments, tools, hardware, and supplies. More specifically the invention relates to a sterile wall that may be quickly assembled and placed along one or more sides of the surgical instrument table thereby preventing the sterile field on the table from being contaminated by unsterilized objects and personnel that approach the table.

### 2. Description of the Related Art

Surgical Site Infections are a leading cause of patient morbidity and mortality in the United States (average 500,000 occurrences a year). Rigorous adherence to the principles of asepsis is the foundation of surgical site prevention and should never be circumvented to save time or money. A sterile field, such as a surgical operating room, should be prepared and maintained for every surgical patient. Included within the sterile field are a variety of items that are to be kept sterile such as surgical instruments, tools, hardware, and supplies for use by the surgeon during surgery. Keeping a sterile field sterile requires careful planning and attention to details. Many techniques are utilized to maintain the sterility of an operating room but with the entrance of humans and surgical equipment into the operating room the probability of contaminants entering the room increases. Even the movement of humans within a operating room should be minimized to reduce the amount of microbial contaminants that may be shed.

While there are many enclosures designed to maintain the sterility of a surgical tray while in storage there is a need to continue to protect the sterility of a surgical tray and other contents on a surgical instrument table while in use. It is very important that the surgical tray not be contaminated during surgery as these contaminants may then be transported to within the body of the patient that is the subject of the surgery. Contaminates from unsterile clothing, packaging, and equipment may come in contact with surgical instruments or other contents on the surgical instrument table and be transported to within the body of the patient. Surrounding the surgical instrument table with a barrier is an approach of preventing contaminants from reaching the surgical instruments, surgical trays, tools, hardware, and supplies that need to stay sterile for a surgical procedure.

US patent publication 2016/0058510 to Blice et al discloses a system for containing used surgical instruments, which includes an instrument container. The instrument container has outer and inner surfaces and a plurality of containment units disposed along the inner surface. The plurality of containment units each covers an area of the inner surface and each has restraints for holding at least one surgical instrument. A foam sheet soaked with a disinfectant compound such as a disinfecting enzymatic liquid that may include bactericidal, fungicidal, virucidal, and/or tuberculocidal properties is used to cover one or more containment units. Such a foam sheet could be used to cover the surgical instruments during surgery but would require that the foam sheet be removed and then returned to its protective covering position each time a surgical instrument is transferred to and from the tray. This is an inconvenience and would likely result in the foam sheet being removed and not returned.

U.S. Pat. No. 4,415,089 to Ruffa discloses a suture and surgical accessory rack includes a frame and bag-like disposable member supported in an upright manner on the frame. The bag-like member has a generally vertically-disposed front panel having secured thereto a multiplicity of pockets wherein surgical instruments may be stored. As each surgical instrument is stored within a pocket the instrument is kept away from airborne contaminants. However as each instrument is stored within a pocket much of the instrument is not visible making it difficult to locate a particular instrument when it is called for by the surgeon.

U.S. Pat. No. 7,104,201 to Comeaux et al discloses a sterile apparatus for covering a surgical tray upon which surgical instruments may be placed. The apparatus comprises two covers over the surgical tray with flaps extending beyond the surgical tray. The flaps may contain pockets to hold surgical instruments similar to the pockets of the Ruffa disclosure and the flaps may also be used to cover and uncover portions of the surgical tray thus covering and uncovering the surgical instruments residing thereon. However, just as with the Ruffa disclosure, the pockets cover much of the surgical instruments making it difficult to locate an instrument being called for. Also like the foam sheet of the Blice disclosure covering and uncovering instruments with the flaps is an inconvenience and would likely result in the flaps being left hanging from the tray and not covering the surgical instruments.

Known devices to protect the sterility of a surgical tray while it is in use lack functionality as they require that (i) the surgical instruments on the surgical tray be hidden under a cover in the form of a sheet or flap thus making it difficult to find a particular instrument and (ii) the cover must continually be removed to locate a remove a surgical instrument and then returned back to its protective rest position thus increasing the likelihood that the cover will not be used during surgery to protect the surgical instruments. It is desirable that a device that protects the sterility of a surgical tray allows for the protection of the surgical instruments while permitting the same to be fully visible and easily retrieved from the surgical tray or returned to the surgical tray.

### BRIEF SUMMARY OF THE INVENTION

The present invention is a flat portion of a sterile material with a certain width and height that is precut in a particular geometry containing one or more grooves to facilitate the folding of the material along certain planes so as to create an assembly of one or three walls that may surround a surgical instrument table and thus providing the surgical instruments, tools, hardware, and supplies within the table a degree of protection from contaminants.

It is an object of the present invention to provide to be easily sterilized, packaged, and stored to occupy a minimal amount of space when not in use, and maintain its sterility while being stored.

It is also an object of the present invention to be easily removed from storage and be quickly deployed in a way that will protect the sterility of surgical instruments, tools, hardware, and supplies positioned on a surgical instrument table.

It is also an object of the present invention that the surgical instruments, surgical trays, tools, hardware, and supplies on the surgical instrument table remain in full view of the individual responsible for removing the surgical instruments and returning them.

Finally it is an object of the present invention that the surgical instruments, instrument trays, tools, hardware, and supplies on the surgical instrument table are provided with an additional degree of protection from contaminants than if the present invention was not in use.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

Neither this summary nor the following detailed description defines or limits the invention. The invention is defined by the claims.

### BRIEF DESCRIPTION OF DRAWINGS

The present invention will become more fully understood from the detailed description and accompanying drawings, wherein:

FIG. 1 is a front view, the back view being the same, of the first embodiment of the present invention in its flat and unfolded configuration suitable for storage.

FIG. 2 is an isometric view of the first embodiment of the present invention in its folded configuration ready to be deployed.

FIG. 3 is an isometric view of the first embodiment of the present invention in its folded configuration deployed with a surgical tray.

FIG. 4 is a front view, the back view being the same, of the second embodiment of the present invention in its flat and unfolded configuration suitable for storage.

FIG. 5 is an isometric view of the second embodiment of the present invention in its folded configuration ready to be deployed.

FIG. 6 is an isometric view of the second embodiment of the present invention in its folded configuration deployed with a surgical tray.

FIG. 7 is a front view, the back view being the same, of the third embodiment of the present invention in its flat and unfolded configuration suitable for storage.

FIG. 8 is an isometric view of the third embodiment of the present invention in its folded configuration ready to be deployed.

FIG. 9 is an isometric view of the third embodiment of the present invention in its folded configuration deployed with a surgical tray.

FIG. 10 is an isometric view of the third embodiment of the present invention in its folded configuration deployed with a surgical tray and containing the deck of the second embodiment.

FIG. 11 is a front view, the back view being the same, of the fourth embodiment of the present invention in its flat and unfolded configuration suitable for storage.

FIG. 12 is an isometric view of the fourth embodiment of the present invention in its folded configuration ready to be deployed.

FIG. 13 is an isometric view of the fourth embodiment of the present invention in its folded configuration deployed with a surgical tray.

FIG. 14 is an isometric view of the fourth embodiment of the present invention in its folded configuration deployed with a surgical tray and containing the deck of the second embodiment.

### DETAILED DESCRIPTION OF THE INVENTION

The following description of the preferred embodiment(s) is merely exemplary in nature and is in no way intended to limit the invention, its application, or uses.

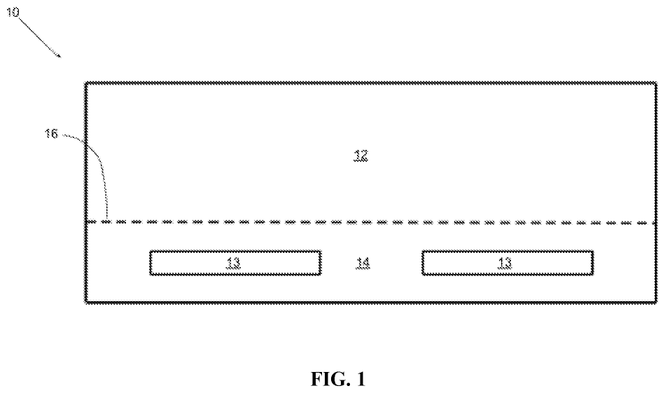

Referring now to the drawings, and more particularly to FIG. 1, a front view of first embodiment 10 of the present invention is shown in its flat and unfolded configuration that is appropriate for storage. The reverse side of first embodiment 10 is identical to the front side with the exception that adhesive strips 13 are missing. First embodiment 10 is made from any material that may be folded and sterilized such as plastic or thick cardstock. To maintain sterility during storage, first embodiment 10 is vacuum wrapped with a plastic wrap of sufficient strength so as not to have sterility compromised during normal handling.

First embodiment 10 is divided into two panels, back 12 and base 14, by guide 16.

Guide 16 is linear channels on the surface of first embodiment 10 to weaken first embodiment 10 along the channel and facilitate folding thereby.

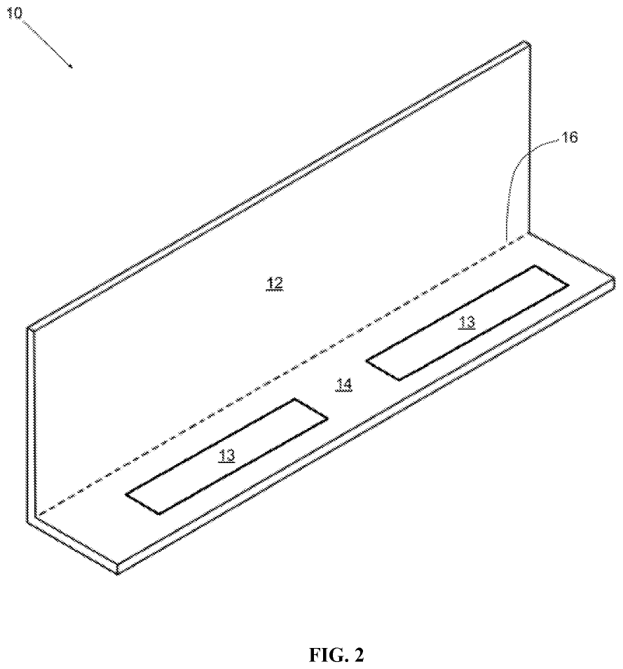

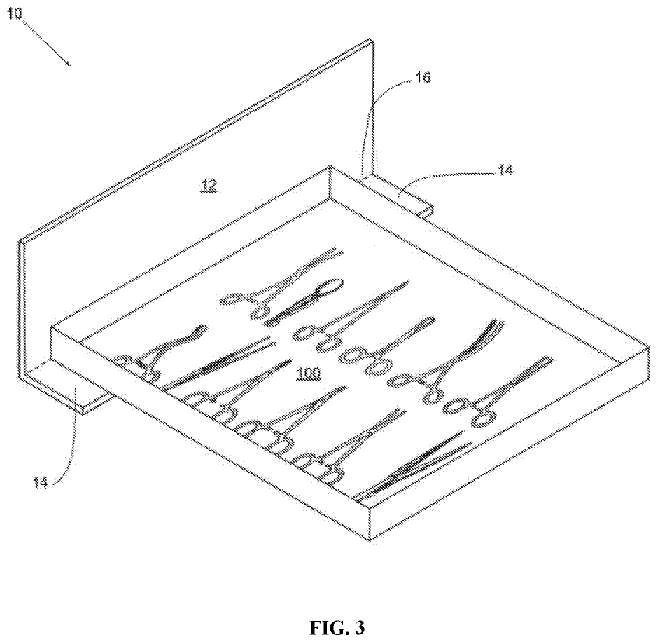

To deploy first embodiment 10 the user will (i) remove first embodiment 10 from storage, (ii) tear off the protective plastic wrap (not shown), (iii) fold first embodiment 10 along guide 16 so that base 14 is perpendicular to back 12 with adhesive strips 13 positioned to receive surgical tray 100 as shown in FIG. 2, and (iv) place the sterile medical apparatus; such as surgical tray 100 that is shown in FIG. 3 or basins, scopes, drills, hardware, stents, and the like; on base 14 so that first embodiment 10 and sterile medical apparatus are releasably joined together by adhesive strips 13.

Clips, well known in the art, may be utilized at both ends of guide 16 to fix at 90 degrees the angle between back 12 and base 14.

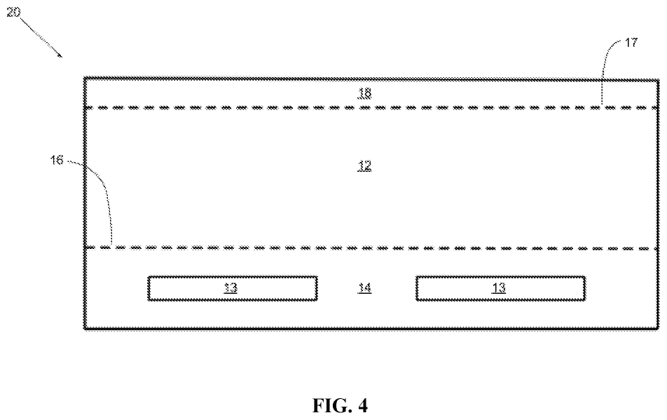

Second embodiment 20 is disclosed in FIGS. 4-6 where FIG. 4 shows a front view of second embodiment 20 in its flat and unfolded configuration that is appropriate for storage. The reverse side of second embodiment 20 is identical to the front side with the exception that adhesive strips 13 are missing. Second embodiment 20 is made from any material that may be folded and sterilized such as plastic or thick cardstock. To maintain sterility during storage, second embodiment 20 is vacuum wrapped with a plastic wrap of sufficient strength so as not to have sterility compromised during normal handling.

Second embodiment 20 is divided into three panels: back 12, base 14, and deck 18. Back 12 and base 14 are separated by guide 16 while back 12 and deck 18 are separated by guide 17. Guides 16 and 17 are linear channels on the surface of second embodiment 20 to weaken second embodiment 20 along the channel and facilitate folding thereby.

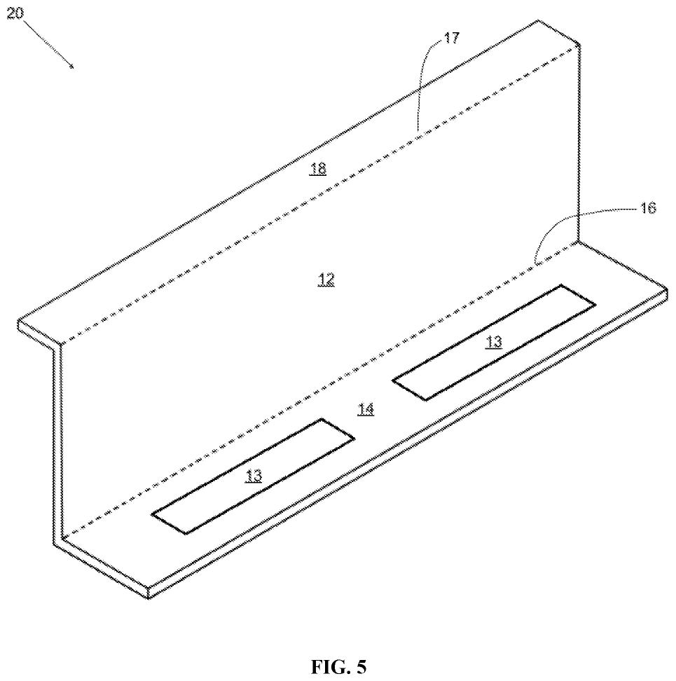

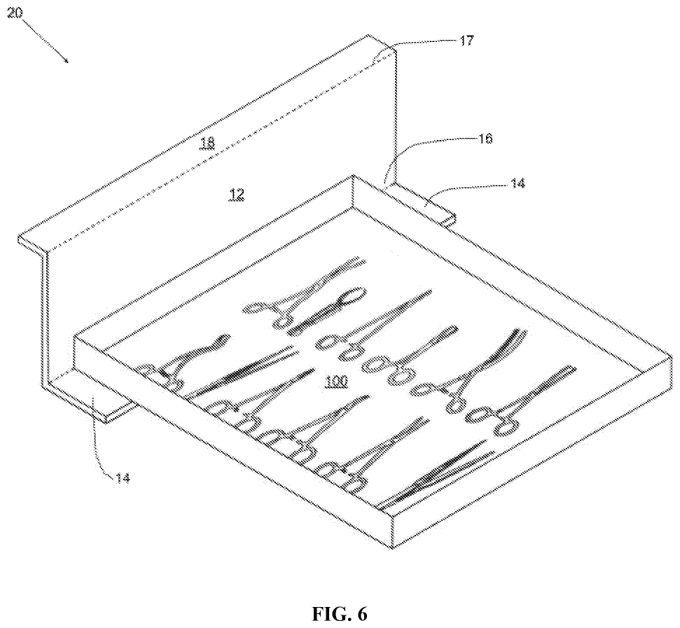

To deploy second embodiment 20 the user will (i) remove second embodiment 20 from storage, (ii) tear off the protective plastic wrap (not shown), (iii) fold second embodiment 20 along guide 16 so that base 14 is perpendicular to back 12 with adhesive strips 13 positioned to receive surgical tray 100 as shown in FIG. 5, (iv) fold second embodiment 20 along guide 17 so that back 12 is perpendicular to deck 18 as shown in FIG. 5, and (iv) place the sterile medical apparatus; such as surgical tray 100 that is shown in FIG. 6 or basins, scopes, drills, hardware, stents, and the like; on base 14 so that second embodiment 20 and sterile medical apparatus are releasably joined together by adhesive strips 13. Clips, well known in the art, may be utilized at both ends of guide 16 and 17 to fix the angular relationship between back 12 and base 14 at 90 degrees as well as the angular relationship back 12 and deck 18 to be minus 90 degrees.

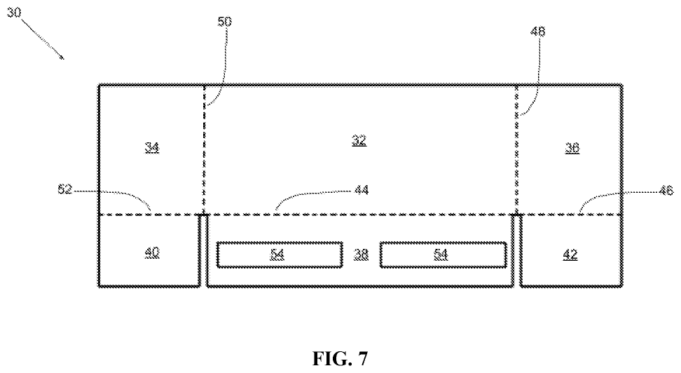

FIGS. 7-9 discloses third embodiment 30 of the present invention which is the preferred embodiment as well. FIG. 7 shows the front view of third embodiment 30 in its flat and unfolded configuration that is appropriate for storage. The reverse side of third embodiment 30 is identical to the front side with the exception that adhesive strips 54 are missing. Third embodiment 30 is made from any material that may be folded and sterilized such as plastic or thick cardstock. To maintain sterility during storage, third embodiment 30 is vacuum wrapped with a plastic wrap of sufficient strength so as not to have sterility compromised during normal handling.

Third embodiment 30 is divided into six rectangular panels in two adjoining rows of three panels each as shown in FIG. 7. The first row contains panels: left side 34, back 32, and right side 36. The second row contains panels: lower left 40, base 38, and lower right 42. Panels left side 34 and back 32 are separated by guide 50. Panels back 32 and right side 36 are separated by guide 48. Panels left side 34 and lower left 40 are separated by guide 52. Panels right side 36 and lower right 42 are separated by guide 46. Panels back 32 and base 38 are separated by guide 44. Panels lower left 40 and base 38 are separated by a gap. Finally, panels base 38 and lower right 42 are also separated by a gap. Guides 52, 44, and 46 form a single linear channel across third embodiment 30 that comes in contact with the gaps separating lower left 40 with base 38 and base 38 with lower right 42. Guides 50 and 48 form linear channels that are perpendicular to guides 52, 44, and 46 and operate from the top of third embodiment 30 to the top of their respective gap. Guides 44, 46, 48, 50, and 52 are linear channels on the surface of third embodiment 30 to weaken third embodiment 30 along the channel and facilitate folding thereby.

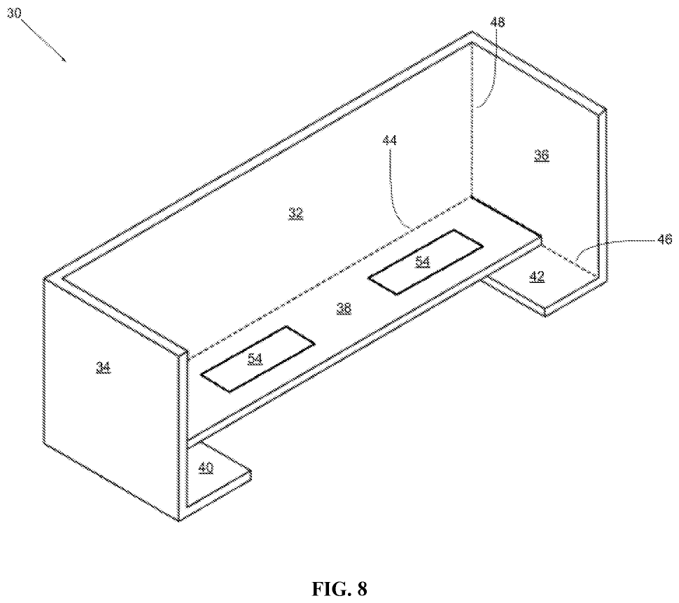

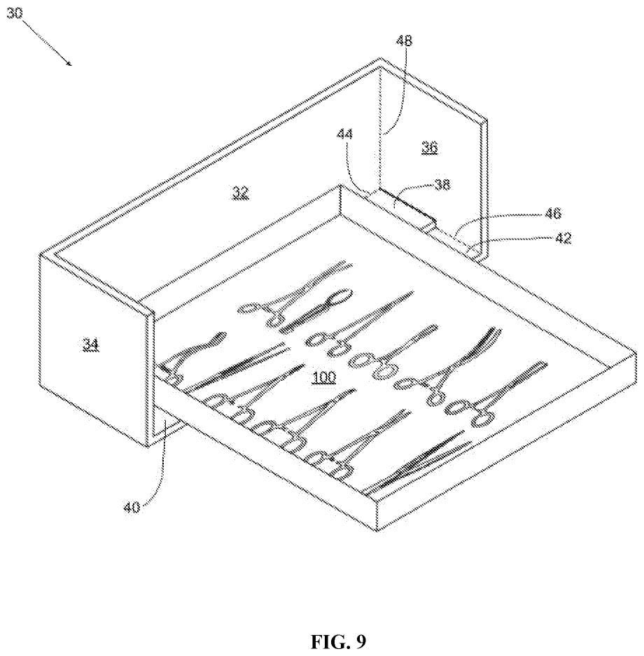

To deploy third embodiment 30 the user will (i) remove third embodiment 30 from storage; (ii) tear off the protective plastic wrap (not shown); (iii) fold third embodiment 30 along guide 44 so that base 38 is perpendicular to back 32 with adhesive strips 54 positioned to receive surgical tray 100 as shown in FIG. 8; (iv) fold third embodiment 30 along guide 50 so that left side 34 is perpendicular to back 32 and abutting base 38 and (v) fold third embodiment 30 along guide 52 so that lower left 40 is under base 38 as shown in FIG. 8; (vi) fold third embodiment 30 along guide 48 so that right side 36 is perpendicular to back 32 and abutting base 38 and (vii) fold third embodiment 30 along guide 46 so that lower right 42 is under base 38 as shown in FIG. 8; and (viii) place the sterile medical apparatus; such as surgical tray 100 that is shown in FIG. 9 or basins, scopes, drills, hardware, stents, and the like; on base 38 so that third embodiment 30 and sterile medical apparatus are releasably joined together by adhesive strips 54. Clips, well known in the art, may be utilized on top of third embodiment 30 at guides 48 and 50 to maintain the perpendicular relationship between left side 34 and back 32 and right side 36 and back 32. Clips may also be utilized on the sides of third embodiment 30 at guides 46 and 52 to maintain the perpendicular relationship between left side 34 and lower left 40 and right side 36 and lower right 42.

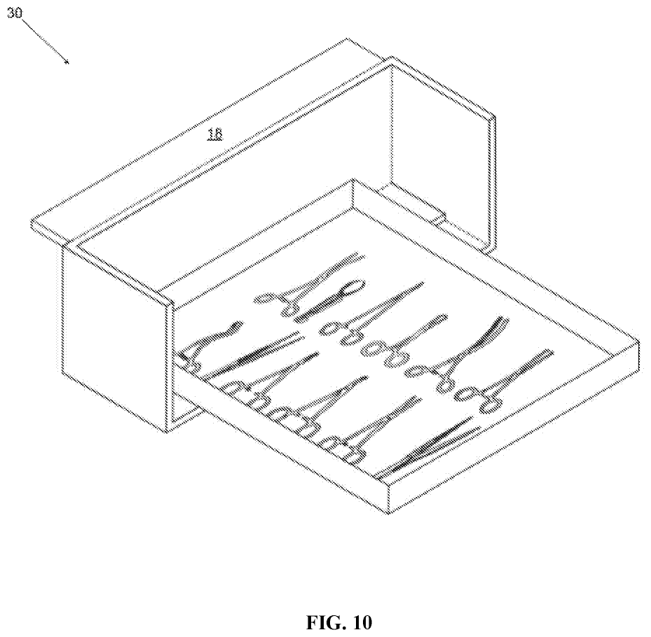

Third embodiment 30 may be further modified by adding deck 18 of second embodiment 20 to the top edge of back 32 as shown in FIG. 10.

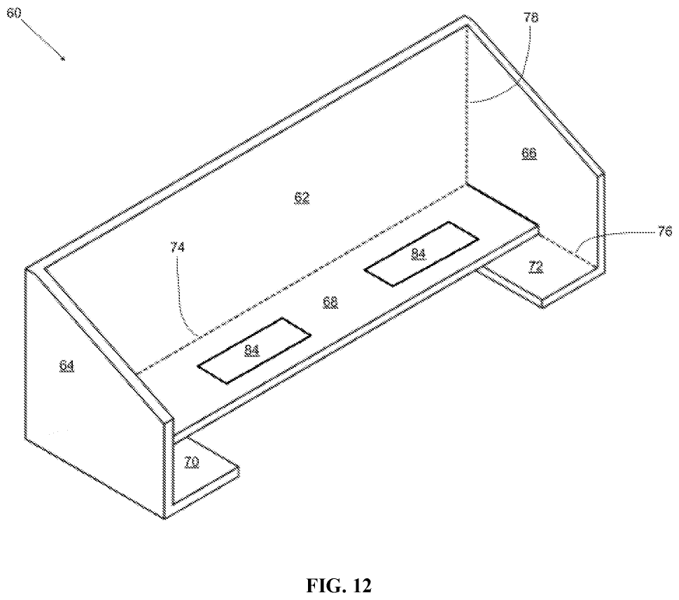

FIGS. 10-12 discloses fourth embodiment 60 of the present invention with FIG. 7 showing the front view of fourth embodiment 60 in its flat and unfolded configuration that is appropriate for storage. The reverse side of fourth embodiment 60 is identical to the front side with the exception that adhesive strips 84 are missing. Fourth embodiment 60 is made from any material that may be folded and sterilized such as plastic or thick cardstock. To maintain sterility during storage, fourth embodiment 60 is vacuum wrapped with a plastic wrap of sufficient strength so as not to have sterility compromised during normal handling.

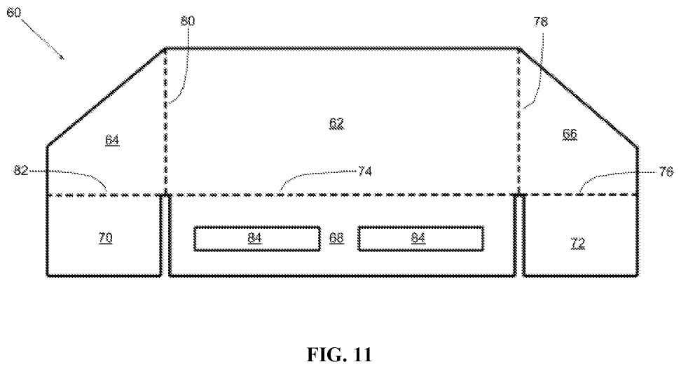

Fourth embodiment 60 is divided into six panels in two adjoining rows of three panels each as shown in FIG. 11. The first row contains panels: left side 64, back 62, and right side 66. The second row contains panels: lower left 70, base 68, and lower right 72. All of the panels are rectangular except for panels left side 64 and right side 66 which are polygons each comprised of a rectangular portion and a triangular portion. Panels left side 64 and back 62 are separated by guide 80. Panels back 62 and right side 66 are separated by guide 78. Panels left side 64 and lower left 70 are separated by guide 82. Panels right side 66 and lower right 72 are separated by guide 76. Panels back 62 and base 68 are separated by guide 74. Panels lower left 70 and base 68 are separated by a gap. Finally, panels base 68 and lower right 72 are also separated by a gap. Guides 82, 74, and 76 form a single linear channel across fourth embodiment 60 that comes in contact with the gaps separating lower left 70 with base 68 and base 68 with lower right 72. Guides 80 and 78 form linear channels that are perpendicular to guides 82, 74, and 76 and operate from the top of fourth embodiment 60 to the top of their respective gap. Guides 74, 76, 78, 80, and 82 are linear channels on the surface of fourth embodiment 60 to weaken fourth embodiment 60 along the channel and facilitate folding thereby.

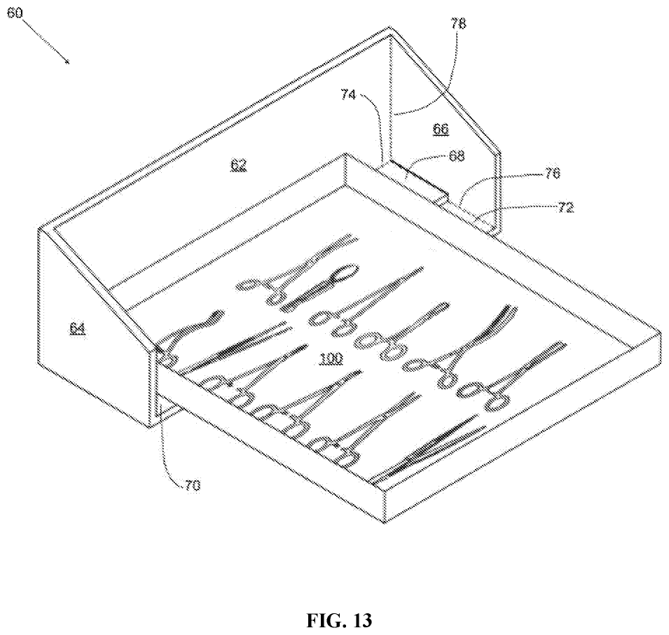

To deploy fourth embodiment 60 the user will (i) remove fourth embodiment 60 from storage; (ii) tear off the protective plastic wrap (not shown); (iii) fold fourth embodiment 60 along guide 74 so that base 68 is perpendicular to back 62 with adhesive strips 84 positioned to receive surgical tray 100 as shown in FIG. 11; (iv) fold fourth embodiment 60 along guide 80 so that left side 64 is perpendicular to back 62 and abutting base 68 and (v) fold fourth embodiment 60 along guide 82 so that lower left 70 is under base 68 as shown in FIG. 11; (vi) fold fourth embodiment 60 along guide 78 so that right side 66 is perpendicular to back 62 and abutting base 68 and (vii) fold fourth embodiment 60 along guide 76 so that lower right 72 is under base 68 as shown in FIG. 11; and (viii) place the sterile medical apparatus; such as surgical tray 100 that is shown in FIG. 13 or basins, scopes, drills, hardware, stents, and the like; on base 68 so that fourth embodiment 60 and sterile medical apparatus are releasably joined together by adhesive 84. Clips, well known in the art, may be utilized on top of fourth embodiment 60 at guides 78 and 80 to maintain the perpendicular relationship between left side 64 and back 62 and right side 66 and back 62. Clips may also be utilized on the sides of fourth embodiment 60 at guides 76 and 82 to maintain the perpendicular relationship between left side 64 and lower left 70 and right side 66 and lower right 72.

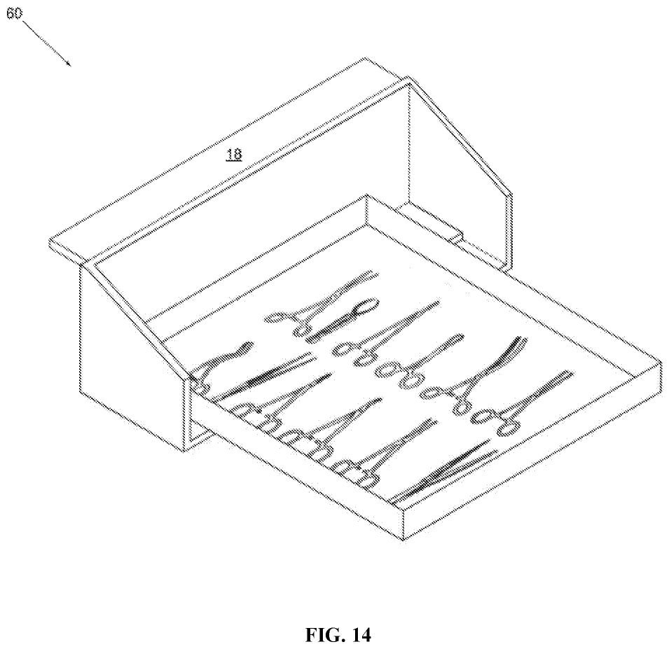

Fourth embodiment 60 may be further modified by adding deck 18 of second embodiment 20 to the top edge of back 32 as shown in FIG. 14.

The above disclosure sets forth a number of embodiments of the present invention described in detail with respect to the accompanying drawings. Those skilled in this art will appreciate that various changes, modifications, other structural arrangements, and other embodiments could be practiced under the teachings of the present invention without departing from the scope of this invention as set forth in the following claims.

1. A device for providing a barrier to contaminants from entering a tray, the device comprising a rectangular sheet having:
a front surface and a back surface;
a top, bottom, left, and right edge;
a width greater than its height and a nominal thickness;
a first row of three rectangular panels along the top edge and occupying the top edge being upper left panel, upper mid panel, and upper right panel with a channel between upper left panel and upper mid panel and a channel between upper mid panel and upper right panel to facilitate folding the material along the channels, the upper mid panel having width equal to or greater than width of the tray;
a second row of three rectangular panels along the bottom edge and occupying the bottom edge being lower left panel, lower mid panel, and lower right panel with a gap between lower left panel and lower mid panel and a gap between lower mid panel and lower right panel to facilitate folding the material along the channels;
the width of lower mid panel is less than or equal to the width of the upper mid panel, the lower mid panel further comprising at least one adhesive patch positioned to be in contact with the tray;
the first row and the second row occupying the height of the material; and
a channel between the first row and the second row to facilitate folding the material.

2. The device of claim 1 further comprising a rectangular sheet of material joined to the top edge of the upper mid panel with a channel separating the rectangular sheet of material from the upper mid panel to facilitate folding the material along the channel.

3. A method of providing a barrier to contaminants entering a tray, the method comprising:
providing a device, the device comprising:
a sturdy sheet, the sheet having a bottom edge,
a pair of fold lines running perpendicular to the bottom edge foldably divide the sheet into three panels, a left panel, a mid panel, and a right panel, the width of the mid panel is equal to or greater than the width of the tray;
a left flap foldably coupled to the left panel at the bottom edge;
a mid flap foldably coupled to the mid panel at the bottom edge;
a right flap foldably coupled to the right panel at the bottom edge;
folding the left panel and the right panel inwards along the fold lines and perpendicular to the mid panel;
folding the mid flap inwards along the bottom edge and perpendicular to the mid panel;
folding the left flap and the right flap towards the mid flap; and
placing the tray over the mid flap such that mid panel forms the barrier.

4. The method of claim 3, wherein the mid flap further comprises at least one adhesive patch, the adhesive patch positioned to be in contact with the tray.

5. The method of claim 3, wherein the mid panel is of rectangular shape, and the left panel and the right panel are of trapezoid shape.

6. The method of claim 3, wherein the mid panel is of square shape, and the left panel and the right panel are of trapezoid shape.

7. The method of claim 3, wherein the tray is a surgical tray for holding surgical instruments.

8. The method of claim 3, wherein the left flap and the right flap are coupled to the mid flap using a plurality of clips.

9. The method of claim 3, wherein the sheet is made of a sterilizable material.

10. The method of claim 3, wherein area of the left panel and area of the right panel is half of the area of the mid panel.

11. The method of claim 3, wherein area of the left panel and area of the right panel is less than half of the area of the mid panel.

12. The method of claim 3, wherein area of the mid flap is less than half of the area of the mid panel.

13. A device for providing a barrier to contaminants entering a tray, the device consisting essentially of:
a mid panel having a width equal to or greater than width of the tray;
a left panel and a right panel foldably coupled to opposite edges of the mid panel, the left panel, the mid panel and the right panel having a continuous bottom edge;
a left flap foldably coupled to the left panel at the bottom edge;
a mid flap foldably coupled to the mid panel at the bottom edge; and
a right flap foldably coupled to the right panel at the bottom edge.

14. The device of claim 13, wherein the left panel and the right panel are of trapezoid shape.
